(19) United States
(12) Patent Application Publication
(10) Pub. No.: US 2002/0018783 A1
(43) Pub. Date: Feb. 14, 2002

(54) FUSION PROTEINS OF A SINGLE CHAIN ANTIBODY AND CD28 AND USES THEREOF

(76) Inventors: MICHEL SADELAIN, NEW YORK, NY (US); NAI-KONG V. CHEUNG, PURCHASE, NY (US); ANJA KRAUSE, NEW YORK, NY (US); HONG-FEN GUO, NEW YORK, NY (US)

Correspondence Address: OPPEDAHL AND LARSON LLP, P O BOX 5068, DILLON, CO, 80435-5068, US

(21) Appl. No.: 08/940,544

(22) Filed: Sep. 30, 1997

Related U.S. Application Data
(63) Continuation-in-part of application No. PCT/US97/04427, filed on Mar. 20, 1997.

(51) Int. Cl. A61K 39/395; A61K 48/00; A61K 39/40; C12P 21/08; C07K 16/00; A61K 39/00; A61K 39/42; A01N 63/00

(52) U.S. Cl. 424/192.1; 424/93.21; 424/141.1; 424/134.1; 530/387.1; 530/387.9

(57) ABSTRACT

Genetically-modified T cells with enhanced survival in vivo are obtained by transducing T cells with a recombinant polynucleotide encoding a fusion protein comprising a single chain Fv antibody (comprising the variable regions of the heavy and light chains of a selected antibody such as an anti-G_{D2} antibody) linked to CD28 receptor. T cells expressing this recombinant fusion protein exhibit enhanced survival when reintroduced to an in vivo environment.. These T cells can be used to induce an immune response to cells, particularly tumor cells, when express the antigen for which the antibody is specific. Cells expressing recombinant fusion proteins according to the invention can also be used for in vitro purging of stem cells/bone marrow and for in vivo targeting of tumor cells and other antigen-bearing cells for purposes of imaging.

This application is a continuation-in-part of International Patent Application No. PCT/US97/04427 filed Mar.20, 1997 designating the United States, which is incorporated herein by reference.

### BACKGROUND OF THE INVENTION

This application relates to a fusion protein incorporating a single chain antibody and the human CD28 receptor. The fusion protein confers antigen-specific recognition and enhanced survival on human polyclonal T-lymphocytes expressing the fusion protein, thus offering the ability to induce an immunological response to a selected antigen by selection of the single chain antibody.

A long-standing goal of cancer research has been to stimulate the immunological rejection of tumors. This goal is based on the hypothesis that many tumors express foreign or mutated forms of antigens that can potentially serve as targets for their destruction by the immune system. Cellular immunity plays the key role in this rejection, with both T helper cells and cytolytic T lymphocytes (CTLs) being involved (Greenberg, 1991; Melief 1992).

There are several reasons why even those tumors that express rejection antigens can evade destruction by T cell immunity. Destruction of immunological targets requires T lymphocytes recognition via the T cell receptor (TCR) of antigenic peptides presented in the context of major histocompatibility complex (MHC) molecules on antigen-presenting cells (APC) (Bjoerkman et al, 1987). Some tumors fail adequately to process and present antigens to T cells because of reduced expression of MHC class I molecules (Elliot et al., 1 989).

Many strategies have been devised to render tumor cells more immunogenic. One is based on the genetic engineering of tumor cells to stimulate the generation of tumor-specific effector T cells in vivo. This has been investigated by direct MHC class I gene transfection to enhance expression of MHC (Hui et al, 1984, Tanaka et al, 1984, Wallich et al, 1985), by introduction of the γ-interferon cDNA to upregulate endogenous class I antigens (Watanabe et al, 1989; Gansbacher et al, 1990a) or by tranfecting tumor cells with cytokines with the hope that interleukin paracrine secretion of lymphokines can substitute for T cell help, induce tumor specific cytotoxic T lymphocytes, and cause tumor rejection (Fearon et al, 1990; Gansbacher et al, 1990b; Ley et al, 1991; Golumbek et al, 1991).

Another approach is based on the manipulation of the effector cells, i.e., T lymphocytes, rather than the antigen-presenting cells or tumor cells. T cells can recognize and lyse tumor cells provided that they bind to the tumor cells and are appropriately activated. T cell activation operates according to the two signal model (Bretscher and Cohn, 1970, Janeway 1989; Nossal, 1989; Schwartz, 1989), which states that lymphocytes require for optimal activation both an antigen-specific signal delivered through the antigen receptor and a second antigen non-specific or costimulatory signal. T cell costimulatory pathways determine whether TCR complex engagement results in functional activation or clonal anergy of CD4⁺ T cells (Mueller et al., 1989; Schwartz 1989).

The molecular basis of T cell costimulation results from an interaction of the T cell surface receptor CD28 with the costimulatory ligand B7, which is primarily expressed on the surface of professional APCs and activated B cells (Liu et Linsley, 1992, Freeman et al, 1989) leading to IL-2 secretion and clonal expansion of the activated T cells (Linsley et al, 1991b; Gimmi et al, 1991; Damle et al, 1992). In vitro and in vivo studies showed that signals transduced by the CD28 receptor determine wether TCR occupancy results in a productive immune response or clonal anergy. (Jenkins et al, 1991; Harding et al, 1992; Linsley et al 1992) Therefore, one factor accounting for the poor immunogenicity of MHC-expressing tumors is that, despite presentation of potentially immunogenic peptides in the context of MHC molecules, tumors lack the costimulatory molecule B7, and thus fail to elicit a full activation of T cells and therefore an effective anti-tumor T cell response. Thus, the introduction of the B7 molecule (CD28 ligand) in tumor cells is one discussed therapy today (melanoma: Townsend et al, 1993; Chen et al, 1992; colon carcinoma: Townsend et al, 1994) to provide protective immunity by autologous CD8⁺ T cells which leads to a potent rejection of modified and unmodified tumor cells in vivo. CD4⁺ and CD8⁺ immunity induced by immunization with class II⁺B7-1⁺- transfected sarcoma cells are also widely discussed immotherapy strategies (Baskar et al, 1995).

An alternative means of generating tumor-specific T lymphocytes is their modification by gene transfer of tumor specific fusion molecules. The introduction of chimeric molecules in T cells combining tumor specific single chain variable fragment (scFv) with signal transduction domains of TCR related activation molecules is reported by a number of groups (Eshar et al, 1993; Hawkins et al, 1996). These genetically modified T cells are able to target tumor cells and to destroy them in vitro, but based on the two signal model for T cell activation, the reinfusion of these transduced T lymphocytes is limited by the incomplete activation signal after antigen recognition and clonal expansion in vivo is not successful .

Thus, there remains a need for a method for sustaining the formation of tumor-specific T lymphocytes which can be successfully reintroduced into a host organism, preferably a human being. It is an object of the present invention to provide such a method, and to provide fusion proteins, and nucleic acid constructs encoding such fusion proteins which can be used in such a method.

As discussed in more detail below, a particularly preferred embodiment of the invention utilizes a fusion protein which combines a single chain antibody to the disialoganglioside G_{D2} and human CD28. Gangliosides are acidic glycosphingolipids found on the outer surface of most cell membranes. Many tumors have abnormal glycolipid composition and structure. Disialoganglioside, G_{D2}, has been found in a wide spectrum of human tumors, including neuroblastoma, osteosarcomas and other soft tissue sarcomas, medulloblastomas, high grade astrocytomas, melanomas, lymphomas and small cell lung cancer. Among glioblastoma multiforme and anaplastic astrocytoma, anti-G_{D2} demonstrated the most restrictive pattern when compared with anti-GD3 and anti-GM2 antibodies.

### SUMMARY OF THE INVENTION

In accordance with the present invention, genetically-modified T cells with enhanced survival and enhanced cytotoxic activity in vivo are obtained by transducing T cells with a recombinant polynucleotide encoding a fusion protein comprising a single chain Fv antibody (comprising the variable regions of the heavy and light chains of a selected antibody such as an anti-G_{D2} antibody) linked to CD28 receptor. T cells expressing this recombinant fusion protein exhibit enhanced survival when reintroduced to an in vivo environment. These T cells can be used to induce an immune response to cells, particularly tumor cells, when express the antigen for which the antibody is specific. Surprisingly, the fusion protein also increases the cytotoxic activity of cytotoxic T lymphocytes against antigen-postive tumor cells.

Cells expressing recombinant fusion proteins according to the invention can also be used for in vitro purging of stem cells/bone marrow. In addition, the recombinant polynucleotide of the invention can be used to preferentially enhance survival of T cells in a mixture of T cells, thus allowing the identification of a minute subset of cells which recognize tumor cells through their T cell receptor.

### BRIEF DESCRIPTION OF THE DRAWING

FIG. 1 shows a map of a recombinant polynucleotide in accordance with invention encoding a single chain antibody and portions of the human CD28 receptor.

### DETAILED DESCRIPTION OF THE INVENTION

This invention relates to recombinant polynucleotides which encode a fusion protein comprising a single chain Fv antibody (scFv), at least the cytoplasmic domain of the human CD28 receptor and a transmembrane domain, which may be the CD28 transmembrane domain or from another source, to the resulting fusion proteins, and to T cells expressing such fusion proteins. scFv-CD28 constructs can be transfected using viral and non-viral vectors into T lymphocytes, particularly human T lymphocytes. Since these scFv are permanently integrated into the cellular genome, retrovirally transduced lymphocytes express scFv on their cell surface and through the CD28 cytoplasmic domain become activated upon antigen binding. scFv facilitates the homing of these cells to tumor sites, thus being effective in promoting both the localization and killing of tumors. With a suicide gene, thymidine kinase or bacterial cytosine deaminase, also transfected, these cells can now be turned on and off as needed.

In the fusion proteins of the invention, the scFv is combined with portions of the human CD28 receptor. The portions of the CD28 receptor included should be sufficient to cytoplasmic domains sufficient to provide signaling function. Portions of extracellular domain and the transmembrane domain from CD28 can also be included, or a transmembrane domain from another source may be used instead. The transmembrane and signaling domains of CD28 can be provided using the portion of the CD28 cDNA spanning nucleotides 336 to 663, which can be amplified by PCR using the primers

GCGGCCGCAA TTGAAGTTAT GTATCCT SEQ ID No. 1 and

TCGAGGATCT TGTCAGGAGC GATAGGCTGC SEQ ID No. 2.

These primers respectively include NotI and BamHI restrictions sites which makes them suitable for insertion in the retroviral Vector SFG. Other primers with other restriction sites can also be used to facilitate use of other vectors.

The antibody portion of the fusion protein of the invention is a construct comprising the variable regions of the heavy and light chains of a selected antibody as a single chain Fv fragment. Single-chain Fv fragments (scFv) offer some of the best opportunities to achieve these results. ScFv technology utilizes molecular biology methods to reduce antibodies to the minimal-required-unit of heavy and light chain variable regions tethered by a peptide linker which can be designed with versatile side chains for radioconjugation. The production of scFv antibodies is described in U.S. Pat. No. 4,946,778 which is incorporated herein by reference.

It will be appreciated by persons skilled in the art that various techniques can be used for constructing the recombinant polynucleotides of the invention which encode scFv-CD28 fusion proteins. In one such technique described in more detail in the examples below, mRNA from a hybridoma producing a monoclonal antibody to the target antigen is purified and reverse transcribed. Amplification of the light and heavy chain regions is then achieved using PCR primers which bind to non-antigen specific portions of the light and heavy chain regions respectively. Kits containing such primers for mouse are commercially available, and these molecules can be humanized if necessary.

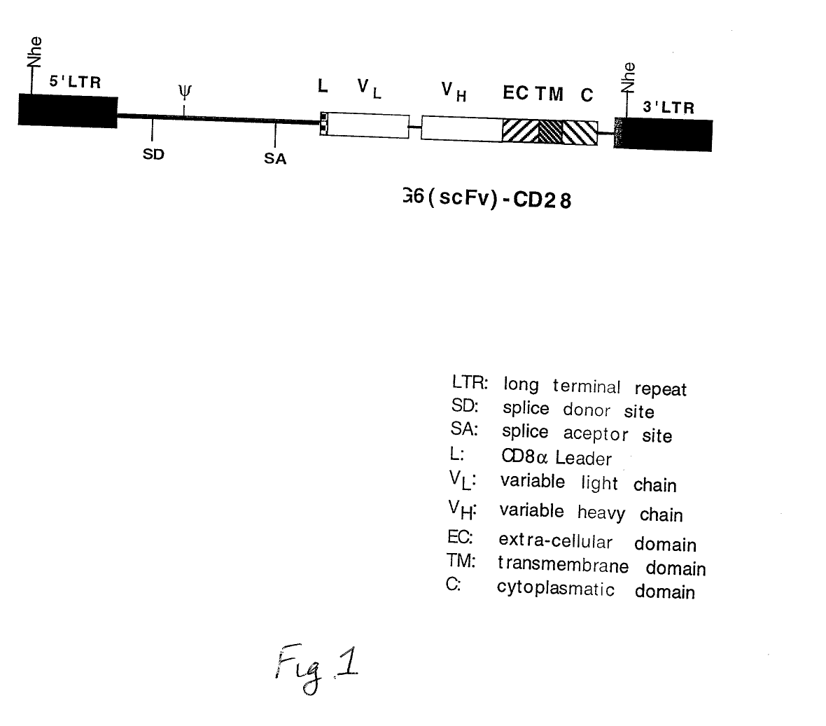

After amplification and recovery of the scFV encoding regions, the resulting cDNA is inserted with the DNA encoding the CD28 receptor into an expression vector which will permit expression of the fusion protein in target cells, particularly human T lymphocytes. FIG. 1 shows a map of one example of a recombinant polynucleotide construct according to the invention. Suitable expression vectors for this purpose include retroviral vectors such as SFG and LXSN, non-viral vectors, and adeno-associated viral vectors.

The recombinant polynucleotide of the invention may also encode additional elements either as part of the fusion protein, for example a second transmembrane domain, or for coexpression with the fusion protein. In particular, it may be advantageous to include a “suicide gene” such as a gene encoding herpes simplex virus thymidine kinase (hsv-tk) or bacterial cytosine deaminase (CDA) in the recombinant polynucleotide. The recombinant polynucleotide may also include a marker gene such as an LNGFR marker or the NTP mutant form thereof which will facilitate recovery of successfully transduced T lymphocytes, whatever the identity of the scFV.

For use in cancer therapy, recombinant polynucleotides are formed which encode single chain Fv antibodies associated with tumor cells. These recombinant polynucleotides are then used to transduce T lymphocytes to produce a population of T lymphocytes which express the fusion protein and any associated proteins also encoded by the recombinant polynucleotide. This procedure will generally be performed ex vivo using peripheral blood lymphocytes from the patient to be treated. After introduction of the recombinant polynucleotide, lymphocytes expressing the fusion protein are reintroduced into the patient. where they target tumor cells bearing the antigen to which the scFv binds. The scFv-CD28 fusion protein on the cell surface of the genetically modified cytotoxic T cells directs antibody-specific anti-tumor T cell responses in vivo. The provision of tumor specificity by this costimulatory fusion molecule bypasses the need for exogenous help from CD4⁺ cells by directly activating CD8⁺ T cells through the antigen recognition and confers enhanced survival in vivo of the activated T cells.

As will be apparent from this discussion, the antibody portions included in the fusion proteins of the invention are selected to target a specific type of tumor. scFv's of interest for incorporation in the invention include scFv's directed against prostate-specific membrane (PSM) antigen, found in prostate cancer cells. A preferred single chain antibody which may be included in the fusion proteins of the invention is an anti-G_{D2} antibody. Cells expressing the fusion protein including the anti-G_{D2} scFV are useful for treatment of neuroblastomas, melanomas, small lung carcinoma, sarcomas and brain tumors which all express G_{d2} as a surface antigen. Anti-G_{D2} scF have been prepared from hybridomas 5F11 and 3G6 using the methods described in the examples, and these. For 5F11 the orientation VH-VL is used for the scFv. For 3G6 the orientation VL-VH is necessary. The sequences of 5F11-scFv and 3G6-scFv are shown below.

| | |
|---|---|
| 5F1 1-scFv | SEQ ID No. 3 |
| CAGGTGAAACTGCAGCAGTCAGGACCTGAACTGGTGNAGCCTGGGGCTTCAG | |
| TGAAGATATCCTGCAAGACTTCTGGANACAAATTCACTGAATACACCATGCAC | |
| TGGGTGAAGCAGAGCCATGGAAAGAGCCTTGAGTGGATTGGAGGTATTAAT | |
| CCTAACAATGGTGGTACTAACTACAAGCAGAAGTTCAAGGGCAAGGCCACAT | |
| TGACTGTAGACAAGTCCTCCAGCACAGCCTACATGGAGCTCCGCAGCCTGAC | |
| ATCTGAGGATTCTGCAGTCTATTACTGTGCAAGAGATACTACGGTCCCGTTTG | |
| CTTACTGGGTCCAAGGGACCACGGTCACCGTCTCCTCAGGTGGAGGCGGTTC | |
| AGGCGGAGGTGGCTCTGGCGGTGGCGGATCGGACATCGAGCTCACTCAGTCT | |
| CCAGCAATCATGTCTGCATCTCCAGGGGAGAAGGTCACCATGACCTGCAGTG | |
| GCAGCTCAAGTATAAGTTACATGCACTGGTACCAGCAGAAGCCTGTCACCTCC | |
| CCCAAAAGATGGATTTATGACACATCCAAACTGGCTTCTGGAGTCCCTGCTCG | |
| CTTCAGTGGCAGTGGGTCTGGGACCTCTTATTCTCTCACAATCAGCAGCATGG | |
| AGGCTGTAGATGCTGCCACTTATTACTGCCATCAGCGGAGTAGTTACCCGCTC | |
| ACGTTCGGTGCTGGGACACAGTTGGAAATAAAACGG | |
| 3G6-scFv | SEQ ID No. 4 |
| AGTATTGTGATGACCCAGACTCCCAAATTCCTGCTTGTATCAGCAGGAGACAG | |
| GGTTACCATAACCTGCAAGGCCAGTCAGAGTGTGAGTAATGATGTGGCTTGG | |
| TACCAACAGAAGCCAGGGCAGTCTCCGAAACTGCTGATATACTCTGCATCCAA | |
| TCGCTACACTGGAGTCCCTGATCGCTTCACTGGCAGTGGATATGGGACGGAT | |
| TTCACTTTCACCATCAGCACTGTGCAGGCTGAAGACCTGGCAGTTTATTTCTG | |
| TCAGCAGGATTATAGCTCGCTCGGAGGGGGGACCAAGCTGGAAATAAAAGG | |
| TGGAGGCGGTTCAGGCGGAGGTGGCTCTGGCGGTGGCGGATCGCAGGTGCA | |
| GGTGAAGGAGTCAGGACCTGGCCTGGTGGCGCCCTCACAGAGCCTGTCCATC | |
| ACTTGCACTGTCTCTGGGTTTTCATTAACCAATTATGGTGTACACTGGGTTCG | |
| CCAGCCTCCAGGAAAGGGTCTGGAGTGGCTGGGAGTAATATGGGCTGGTGG | |
| AAGCACAAATTATAATTCGGCTCTTATGTCCAGACTGAGCATCAGCAAGGACA | |
| ACTCCAAGAGCCAAGTTTTCTTAAAAATGAACAGTCTGCAAACTGATGACACA | |
| GCCATGTACTACTGTGCCAGTCGGGGGGGTAACTACGGCTATGCTTTGGACT | |
| ACTGGGGTCAAGGAACCTCAGTCACCGTCTCCTCA | |

When a fuision molecules combining GD₂ tumor specificity through a genetically engineered scFv and costimulatory signal the CD28 cytoplasmic domain, the full costimulatory signal for T cell activation and survival is triggered by recognition of tumor cells expressing the GD₂ antigen. Furthermore, activated, primary CD8 ⁺ T cells transduced with the recombinant polynucleotide of the invention are able to lyse G_{D2}-positive tumor cells. Thus the recombinant polynucleotides of the invention can be used in therapy to induce in a host organism an immune response to tumor cells expressing a surface antigen comprising the steps of

(a) transducing T cells to introduce an expressible recombinant polynucleotide encoding a fusion protein comprising the variable region of the light chain of an antibody against the surface antigen, linked to a region encoding the variable region of the heavy chain of an antibody against the surface antigen and a region encoding human CD28 receptor; and

(b) introducing transduced T cells expressing the recombinant polynucleotide into the host.

Lymphocytes and other leukocytes expressing fusion proteins in accordance with the invention can also be used in vitro for purging stem cell/bone marrow preparation. Cytotoxic T cells or NK cells, including lymphokine activated killer (LAK)cells expressing scFvCD28 fusion protein can be added to a patient's phoresis blood sample to kill tumor cells which may be present, and thus to reduce the risk of reintroducing cancer when the phoresis blood is returned to the patient.

### EXAMPLE 1

5F11 hybridoma cells were processed for mRNA using a commercially available kit (Quick Prep Micro mRNA Purification, Pharmacia Biotech) following the procedures outlined by the manufacturer. Briefly, hybridoma cells were cultured in ROPMI- 1640 medium supplemented with 10% calf serum, 2 mmol/L L-glutamine (Sigma), 100 U/L penicillin and 100 ug/ml streptomycin sulfate (Sigma). The cell cultures were maintained at 37° C. under a water-saturated atmosphere of 5% CO₂. 5×10⁶ cells were pelleted by centrifugation at 800 g and washed once with RNase-free phosphate buffered saline (pH 7.4), The recentrifuged cells were lysed directly in the extraction buffer. Poly(A)+RNA was purified by a single fractionation of oligo(dT)-cellulose and then eluted with elution buffer. The mRNA sample was precipitated for 1 hour at 100 ug glycogen, 40 ul of 2 M potassium acetate and 1 ml absolute ethanol at −20° C. The nucleic acids were recovered by centrifugation at 10,000 g for 30 minutes, The sample was evaporated until dry and dissolved in 20 ul RNase-free water.

The mRNA preparation was used in the construction of the 5F11 scFv gene using the Mouse ScFv Module/Recombinant Phage Antibody System (Pharmacia Biotech). 5 ul of the mRNA preparation was reverse transcribed in a total volume of 11 ul of reaction mixture and 1 ul DTT solution for 1 hour at 37° C. For PCR amplification of immunoglobulin variable region, light primer mix and the heavy primer set were added respectively to generate quantities of the light (325 bp ( and heavy (340 bp) chains. Following an initial 5 minute dwell at 95° C., 5 U Ampli Taq DNA polymerase (Perkin Elmer) was added. The PCR cycle consisted of a 1 min denaturation step at 94° C., a 2 min. annealing step at 55° C. and a 2 min extension step at 72° C. After 30 cycles of amplification, PCR derived fragments were purified using glassmilk beads (Bio 101 Co.) and evaluated by electrophoresis on 1/5% agarose gel in TAE buffer with ethidium bromide visiualization. For the assembly and fill-in reaction, both purified heavy chain and light chain fragments were added to ab appropriate PCR mixture containing linker-primer, dNTPs, PCR buffer and Ampli Taq DNA polymerase. Denaturation was performed at 94° C. for 1 minute, followed by a 4 minute annealing reaction at 63° C. The heavy and light DNA were joined into a single chain with linker DNA after 7 thermocycles. Using this single chain DNA as a template and restriction site primers (RS primers) containing either SfiI or NotI restriction sites, secondary PCR amplification was carried out for 30 cycles to amplify the ScFv DNA and add the restriction sites. This introduced the SfiI restrictions site at the 5′-end of the heavy chain and the NotI restriction site at the 3′-end of the light chain. Amplified ScFv DNA was then purified by glassmilk beads and digested with SfiI and NotI .

Purified scFv DNA was inserted into the pCantab 5e vector (Pharmacia Biotech) by ligation as SfiI/Notl sites in the vector. Competent E. coli XL 1-Blue cells were transformed with pCantab 5E phagemid containing the ScFv DNA following the method outlined in Stratagene protocols. For rescue of a recombinant phage antibody library, the helper phage M13 K07 was added.

Antibody-producing recombinant phage were selected by panning using the method of Ditzel, PNAS USA 91: 3710-3714(1994) with slight modifications. 20 ul of GD2(1 ul/ml) dissolved in ethanol were directly coated on a 96-well polystyrene plate and dried at rom temperature. Then 100 ul of the supernatant containing the phage library was added to each well and incubated for 2 hours. The plate was then washed 10 times with PBS containing 0.05% BSA to remove nonspecifically bound phage. Antibody-positive recombinant phage captured by the GD2 antigen was eluted with 0.1 M HCl (pH 2.2 with solid glycine and 0.1% BSA) and neutralized with 2M Tris solution. Selected phage was then re-panned for two additional cycles to further enrich the GD2-binding recombinant phages.

The selected phage was used to reinfect E coli XL1-Blue cells. Clones were grown in 2XYT medium containing ampicillin (100 ug/ml) and 1% glucose at 30° C. until an OD₆₀₀ of 0.5 was obtained. Expression of ScFv antibody was induced by changing to a medium containing 100 uM IPTG and incubating overnight at 30° C. The supernatant obtained from the medium by centrifugation was directly added to a plate coated with GD2. The pellet was resuspended in PBD containing 1 mM EDTA and incubated on ice for 10 minutes. The periplasmic soluble antibody was collected by centrifugation again and added to the plate. After incubating at 37° C. for 32 hours, anti-E Tag antibody (Pharmacia Biotech) was used to specifically screen the binding of the ScFv fragment.

For construction of the SFpoStMCH vector which contains the 5F11-scFV-streptavidin plasmid DNA, plasmid DNA from the 5F11-scFv in pCantab 5E vector (Pharmacia Biotech) was purified and amplified by PCR using two specially designed primers S6 and 318s. S6 contains a NotI restriction site and 318s contains a PvuII restriction site so that amplified DNA can be restriction digested and inserted in the pSTE vector (Dr. Dubel, German Cancer Center). The resulting vector 5FpoStMCH is the 5F11-scFv-streptavidin construct. The streptavidin was digested with BamHI, leaving the scFV 5FpoMCH.

### EXAMPLE 2

In constructing 3G6-scFv, the orientation VH-VL did not produce a functional scFV. Therefore the orientation VL-VH was used. cDNA of VH and VL of 3G6 hybridoma were linked through a custom built linker and inserted into the pHEN vector (Dr. Greg Winter). NcoI and NotI restriction sites were built into the VH and VL linkers so that the scFV can be digested with these enzymes for insertion in the pSTE vector. Clone 7 was chosen and called 3GpoStMCH.

### EXAMPLE 3

To produce a cell-surface molecule capable of CD28 signaling in T cells interacting with G_{D2}⁺ tumors, we constructed a chimeric molecule containing the antigen-binding site of a G_{D2}-specific antibody and the transmembrane and signaling domains of the CD28 molecule as shown in FIG. 1. Several scFv domains derived from the monoclonal antibody 3G6 (Ye et al., 1992) were cloned and tested for their specificity for the G_{D2} antigen. The scFv 3G6.1 cDNA was fused to the human CD8α leader sequence and a segment of the human CD28 cDNA that encodes part of the extracellular, the transmembrane, and the cytoplasmic domains. This segment of cDNA was amplified by PCR from the plasmid pbsCD28, using the upstream primer

5′GCGGCCGCAATTGAAGTTATGTATCCT SEQ ID No. 1

and the downstream primer

5′TCGAGGATCTTGTCAGGAGCGATAGGCTGC SEQ ID No. 2.

These primers contain NotI and BamHI sites respectively for the insertion of the PCR product in the retroviral Vector SFG. Following digestion of the purified PCR product with NotI and BamHI, the CD28 fragment was ligated into the NotI an BamHI sites of the retroviral vector SFG, containing the CD8α leader sequence, followed by the single chain gene, encoding the V_{H} and V_{L} domains of the G_{D2} specific antibody. This molecule, termed 3G6-CD28, was cloned into the retroviral vector SFG (Riviere et al., 1995), as shown in FIG. 1. A truncated form 3G6-CD28TR was cloned as well by PCR, using a downstream primer, which creates 9 basepairs after the transmembrane domain a stop codon.

The chimeric 3G6-CD28 gene was first expressed in NIH 3T3, a murine fibroblast cell line, and in Jurkat, a human leukemia cell line, to characterize the structure of the fusion protein. Retroviral-mediated gene transfer was carried out using high titer producer cells generated by crossinfection of PG13 packaging cells with supernatant harvested from the ecotropic packaging cell line BOSC or by transfection of 293GPG cells with 10 μg SFG/3G6-CD28, SFG/3G6-CD28TR or SFG-NTP DNA, as described (Riviere and Sadelain, 1996). NIH 3T3 and Jurkat were infected with the retroviral particels derived from the transient transfected BOSC/3G6-CD28 or 293GPG/3G6-CD28 in the presence of 8 μg/ml polybrene, respectively. Expression were monitored after 72 hours by FACS analysis and positive subclones were obtained in a limiting dilution assay.

Individual PG13/3G6-CD28 clones were expanded and tested for gene transfer in A549 (obtained from American Tissue Type Collection (ATCC), Rockville, Md., USA). The clone with the highest concentration of infectious particles were further used for retroviral infection in peripheral blood lymphocytes (PBL) (Gallardo et al.,1996). Peripheral blood mononuclear cells (PBMC) from individual healthy donors were separated on Ficoll and activated with phytohemagglutinin A (PHA) at 0.5 μg/ml (Murex Diagnostics, Norcross, Ga., USA) in complete RPMI 1640 medium (Cellegro) containing 10% heat-inactivated AB pooled human serum (Pel Freez Biologicals, Brown Deer, Wis., USA). After 48 hours cells were cocultivated for 18 hours with the relevant producer cells in the presence of polybrene (4 μg/ml). The next day, cells were resuspend in medium containing 10% Fetal Calf Serum (Sigma) and 10 U/ml IL-2 (Cetus,).

After 3 days, gene expression was measured by FACS analysis which confirmed the presence of the fusion protein on the cell surface. The transduced NIH 3T3 and Jurkat cells were stained 72 hours after retroviral infection with the anti-idiotypic antibody A1G4. The chimeric molecule was detected on the cell surface of 91% of the NIH 3T3 cells and 92% of the Jurkat cells. Noninfected cells showed a background stain of less than 2%, thus establishing that 3G6-CD28 was expressed on the cell surface. This phenotype was stable as reflected by the unchanged fraction of positive cells after several weeks in culture, further indicating that expression of scFv by Jurkat cells was neither toxic nor advantageous. As shown by Southern blot analysis, the Jurkat cells were transduced with the intact proviral structure, establishing that the vector SFG-3G6-CD28 was stably integrated and did not rearrange.

To confirm the expression of a full length protein and its ability to dimerize, a Western blot was performed under either reductive or nonreductive conditions. As the fusion protein 3G6-CD28 spans the dimerization domain of CD28 (Arrufo et al., 1987; Ledbetter et al., 1988), we expected to obtain a homodimer of 72 kDa, excluding post-translational modifications. Heterodimers of 3G6-CD28 and endogenous CD28 could also be expected in the transduced Jurkat cells, but not in the fibroblasts. The cell lysates obtained from the NIH 3T3 cells and Jurkat cells were either used as a whole extract or after immunoprecipitation with the AIG4 antibody. Under reducing conditions, the polyclonal anti-CD28 antibody detected in the extract of 3G6-CD28 transduced NIH 3T3 a molecule of 36 kDa, as expected, and in the extract of transduced Jurkat cells a molecule of ca. 40 kDa. The increased molecular weight observed in Jurkat cells can be attributed to post-translational modifications, presumably a potential N-linked glycosylation site.

### EXAMPLE 4

The engagement of CD28 by its natural ligand on antigen presenting cells, B7, results in a costimulatory signal in Jurkat cells and normal T cells characterized by the phosphorylation of tyrosines in a restricted number of proteins (Vandenberghe et al., 1992; Lu et al., 1992) that include the CD28 receptor itself (Pages et al., 1994; Truitt et al.,1994; Prasad et al.,1994; Hutchcroft et al., 1994) and the catalytic subunit of the PI₃-kinase (Lu et al., 1994). The tyrosine phosphorylation of CD28 at tyrosine¹⁷³ is critical for the association of CD28 with the 85 kDa regulatory subunit of the PI₃-kinase which is an essential proximal activation event in the CD28 signaling pathway (Pages et al., 1994; Truitt et al 1994, Stein et al., 1994).

As CD28 signaling can be triggered by anti-CD28 antibodies (Vandenberghe et al., 1992; Lu et al., 1992), we investigated whether crosslinking of 3G6-CD28 with the anti-idiotypic AIG4 Mab could induce the typical CD28-dependent phosphorylation pattern and the binding of the p85 PI₃-kinase subunit. Postnuclear supernatants were first analyzed by an anti-phosphotyrosine blot. Within 5 minutes, crosslinking of the fusion molecule on the surface of the 3G6-CD28/Jurkat clone caused the tyrosine-phosphorylation of proteins of 110/97 kDa, 70 kDa and 36 kDa. The 110/97 and 70 kDa phosphoproteins are also observed in response to crosslinking of the endogenous CD28 with the anti-CD28 Mab 28.2 and GαMIg. Activation of a control Jurkat cell clone with AIG4 and GαRIg did not show the activated pattern, thus establishing that tyrosine phosphorylation was selectively induced by the crosslinking of 3G6-CD28. Immunoprecipitation of the 3G6-CD28 receptor by A1G4 plus GαRIg resulted in an immunocomplex of 3G6-CD28 and the p85 subunit of the PI₃-kinase, as shown by the anti-p85 immunoblot. As for the phosphotyrosine blot, no immunocomplex could be detected in the control lanes. An identical pattern of p85 association with endogenous CD28 was observed after crosslinking of the endogenous CD28 with the 28.2 Mab.

From these data, we conclude that the earliest events in the CD28 pathway are activated in response to 3G6-CD28 crosslinking, suggesting that the engagement of 3G6-CD28 can fully mimic the effect of the endogenous CD28 molecule.

### EXAMPLE 5

We next investigated the CD28 function of 3G6-CD28 in the later events of T-cell activation elicited by CD28 engagement. CD28 signaling causes an increase in the steady state level of IL-2 mRNA by inducing the binding of a nuclear protein complex to the IL-2 promoter (Fraser et al., 1991; Lu et al., 1992) and by increasing IL-2 mRNA stability (Lindsten et al., 1989). This results in an increased IL-2 production by activated T lymphocytes (June et al., 1987; Thompson et al., 1989; Jenkins et al., 1991; Lu et al., 1994). Previous studies have shown that two biochemical signals, provided by a phorbol ester (e.g., PMA) and a calcium ionophore (ionomycin), lead to IL-2 production which is enhanced five-to seven-fold by engagement of the CD28 receptor with anti-CD28 Mab (Thompson et al., 1989). The cytoplasmic domain of CD28 is necessary and sufficient for this costimulation event (Gmunder et al., 1984; Stein et al., 1994; Hutchcroft et al., 1995).

We therefore investigated whether crosslinking by the anti-idiotypic antibody A1G4 could potentiate the IL-2 secretion elicited by PMA and ionomycin in cloned 3G6-CD28 Jurkat cells. 3G6-CD28- and NTP-transduced Jurkat were thus stimulated with the soluble form of anti-idiotype Mab A1G4 or CD28.2 in combination with PMA and ionomycin or with PMA/ionomycin alone. After 24 hours, IL-2 concentration was measured in the culture supematants by ELISA. PMA plus ionomycin alone induced IL-2 secretion in both Jurkat-NTP and Jurkat-3G6-CD28 (290 and 400 pg/ml, respectively). Addition of the anti-CD28 Mab 28.2 increased this secretion by a factor of three in both cell populations (960 and 1200 pg/ml). The addition of A1G4 antibody to Jurkat-3G6-CD28 had a comparable effect (about a five-fold increase of IL-2 production), but no effect on mock transduced Jurkat cells. Thus, these results further suggest that 3G6-CD28 crosslinking is able to provide the downstream signaling events normally elicited by CD28.

### EXAMPLE 5

In order to study the function of 3G6-CD28 in relevant target cells, producer cell lines and culture conditions were established for efficient gene transfer in primary T lymphocytes. High-titer clones encoding 3G6-CD28 and 3G6-CD28TR were generated from the PG13 packaging cell line (Miller et al., 1991) as described elsewhere (Gallardo et al., 1997). 3G6-CD28TR is a control molecule derived from 3G6-CD28 by truncation of the cytoplasmic tail and therefore unable to be phosphorylated or to associate with the p85 subunit of the PI₃-kinase. The previously described PG13/NTP6 clone was used as an irrelevant control, and encodes the cell surface marker NTP, an inactive mutant of the human low-affinity nerve growth factor receptor (Gallardo et al., 1997). Peripheral blood mononuclear cells (PBMC) were exposed to retroviral particles 48 hours after PHA activation, as described in Materials and Methods. The transduced T cells were stained 3 days later with an anti-CD8 antibody and either A1G4 to stain 3G6-CD28 and 3G6-CD28TR or the 20.4 antibody to stain NTP (Gallardo et al., 1997). The infection of PBL yielded between 28 and 40% positive T cells after cocultivation with the producers PG13/3G6-CD28-12, PG13/3G6-CD28TR, and PG13/NTP6. As reported earlier (Gallardo et al., 1997), a comparable fraction of both CD4+ and CD8+ subsets were transduced. The three groups of cells expanded the same rate as the untransduced cells maintained under identical conditions (data not shown). Gene transfer was authenticated by Southern blot analysis which indicated that the cells positive for staining with A1G4 had on average one integrated vector copy per cell.

### EXAMPLE 6

To examine whether 3G6-CD28 could enhance survival of T lymphocytes under conditions where T cell receptor stimulation causes apoptotic cell death, we took advantage of a model system in which T cells stimulated with anti-CD3 Mab undergo apoptosis that can be prevented by CD28 signaling (Radvanyi et al,1993; Shi et al, 1995; Boise et al, 1995). It has been previously reported that activated T cells undergo apoptosis within 48 hours of crosslinking their T cell receptor in a dose dependent manner in the presence of IL-2, when they are reactivated in a non resting stage (Russel et al, 1991; Groux et al, 1993). T lymphoblasts in general, maintained in culture in the presence of low dose of IL-2 (10 U/ml) every 3 days, express the IL-2R p55 chain on their surface and proliferate in the absence of CD3 stimulus. However this low dose of IL-2 is unable to prevent CD3-mediatrd T cell death (Groux et al, 1993; Liu et al, 1990). We therefore investigated whether crosslinking of 3G6-CD28 by the A1G4 Mab could substitute for CD28 stimulation and rescue transduced primary T cells from CD3-induced cell death. The primary T lymphocytes, transduced with NTP, 3G6-CD28, or 3G6-CD28TR, were kept for three days under one of the following conditions: medium alone—which included IL2 at 10 U/ml to prevent cell death due to cytokine deprivation—, medium and OKT3—to trigger apoptosis—, medium and OKT3 and CD28.2—to nonspecifically prevent apoptotic cell death in in all three T cell populations—, and medium and OKT3 and A1G4—to evaluate whether a specific survival would be conferred upon the transduced cells—. In the latter case, the untransduced cells serve as an internal control and the selective survival of the transduced cells would increase the relative percentage of transduced cells in the culture. In contrast, in all other groups and in the cells transduced with 3G6-CD28TR or NTP, the percentage of transduced cells would not be affected by the different culture conditions. After three days, the T cells were released from the crosslinking conditions and kept for two days in the presence of medium and 10 U/ml IL2. This five day procedure was repeated twice.

The survival effect of 3G6-CD28 crosslinking on TCR-mediated apoptosis were first determined by trypan blue staining. Table 1 shows the percentage of dead cells to alive cells (D/A) and the percentage of dead cells (respectively) and represents 1 of 3 independent experiments, counted on day 3 and day 6. The addition of OKT3 to all three groups resulted after 6 days in an 70% increase of dead cells. Similar results were observed when NTP- and 3G6-CD28TR-transduced T cells were incubated with A1G4. A increase of dead cells to only 32% after 3 days and to 13% after 6 days was measured in the 3G6-CD28 T cells. Survival in all three groups was comparable in the presence of anti-CD3 and anti-CD28 antibodies. Thus, A1G4 was able to promote the survival of 3G6-CD28 transduced T cells under in vitro conditions favoring apoptotic cell death.

To demonstrate that survival was a direct consequence of expressing 3G6-CD28, we examined whether the fraction of live cells (68% on day 3 and 87% on day 6) expressing 3G6-CD28, but not 3G6-CD28TR or NTP, increased as a result of preferential survival. The fraction of 3G6-CD28-,3G6-CD28TR-, and NTP-positive T cells was enumerated by FACS analysis (Tab. 2). Under all four conditions the percentage of the control groups NTP and 3G6-CD28TR remained constant, about 30% positive. As expected, the 3G6-CD28 transduced T cells also remained in the 30% range of T cells cultured in either medium alone, OKT3, or OKT3 and anti-CD28 antibodies. When cultured in the presence of OKT3 and A1G4 antibodies, the transduced cells increased from 30% on day 0 to 60% on day 6 to 88% on day 12.

Thus, the data obtained in Jurkat T cells and in peripheral blood lymphocytes establish that the engagement of the 3G6-CD28 fusion molecule with the anti-idiotypic antibody A1G4 provides the costimulatory signals for IL-2 secretion and the prevention of TCR-induced apoptosis normally afforded by the CD28 cytoplasmic tail.

TABLE 1
Survival of 3G6-CD28 transduced T lymphocytes under apoptosis
inducing conditions. NTP, 3G6-CD28 and 3G6-CD28TR transduced T
lymphocytes were cultured for three days in medium alone, with CD3,
CD3 and CD28, and CD3/A1G4. On day 3 (d3) and day 6 (d6) cells were
counted, differentiating dead from alive by trupan blue staining. The
numbers represent the % of dead cells of each population on these two
time points.

| | Medium | | OKT3 | | OKT3/28.2 | | OKT3/A1G4 | |
|---|---|---|---|---|---|---|---|---|
| | d3 | d6 | d3 | d6 | d3 | d6 | d3 | d6 |
| NTP | 15 | 25 | 55 | 76 | 12 | 15 | 55 | 73 |
| 3G6-CD28 | 18 | 22 | 42 | 70 | 10 | 12 | 32 | 13 |
| 3G6D28TR | 19 | 20 | 45 | 70 | 12 | 12 | 50 | 76 |

### EXAMPLE 7

To assess whether 3G6-CD28 confers upon T cell the ability to recognize the G_{D2} antigen, we opted to examine whether transduced CD8 ⁺ T cells, activated by CD3 stimulation, could lyse G_{D2}⁺ target cells in an MHC-unrestricted fashion. 3G6-CD28 and NTP transduced T cells were depleted of CD4 ⁺ cells by magnetic beads. The nonadherent fraction was mostly comprised of CD8⁺ lymphocytes (up to 96% CD8 ⁺ cells) and retained, as expected, the same fraction of NTP⁺ and 3G6-CD28⁺ cells as the unseparated T cells. These purified cytotoxic T cells were further stimulated over 4-6 days with IL-2 (100 U/ml) and OKT3 (10 ng/ml). Prior to setting up the cytotoxicity assays, the T cells were released from OKT3 activation by changing the medium for 24 hours to decrease unspecific killing. Cytotoxic activity was measured against various G_{D2}⁺ target cells in a standard 4 hr ⁵¹Cr release assay. The human neuroblastoma cell lines LAN-1 and LAN-15N, melanoma cell line HTB67 all express camparable and high amounts of the ganglioside G_{D2} (FACS analysis, data not shown) and were used as targets.

In a dose-related fashion, 3G6-CD28 transduced CTLs display consistantly a lytic activity toward the human tumor targets. At a ratio of 25:1 (effector:target) the presence of the 3G6-CD28 molecule on the gene modified T cells provides targeting and lysing activities on up to 30% on HTB67(Sk-Mel-1), near 80% on Lan-15N, up to 60% on LAN-1. CTL activity in the control group was minimal, less than 5% at this ratio. To confirm that cytotoxicity was specifically dependent on the recognition of the GD2 antigen, we performed an inhibition experiment using G_{D2}Dspecific F(abÕ)₂ derived from the antibody 3F8 (Cheung et al, 1985). The lysis of the LAN-1 target cell was blocked by the G_{D2}-specific F(abÕ)₂. Between the effector-to-target ratios of 25:1 to 3: 1, cytotoxicity was reduced from 60% to 20% without antibody to background levels in the presence of the F(abÕ)₂.

From this result we conclude that 3G6-CD28 can be engaged not only by the anti-idiotypic antibody A1G4, but also by antigen expressed on the surface of tumor cells, irrespective of their MHC haplotype.

1. A recombinant polynucleotide encoding a fusion protein comprising the variable region of the light chain of a selected antibody linked to the variable region of the heavy chain of the selected antibody, the signaling domain of human CD28 receptor and a transmembrane domain.

2. The recombinant polynucleotide of claim 1, wherein the transmembrane domain to the human CD28 transmembrane domain.

3. The recombinant polynucleotide of claim 1, wherein the selected antibody is an anti-G_{D2} antibody.

4. The recombinant polynucleotide of claim 3, further comprising a region encoding a suicide gene.

5. The recombinant polynucleotide of claim 4, wherein the suicide gene encodes thymidine kinase.

6. The recombinant polynucleotide of claim 1, further comprising a region encoding a suicide gene.

7. The recombinant polynucleotide of claim 6, wherein the suicide gene encodes thymidine kinase.

8. A recombinant peptide comprising the variable region of the light chain of a selected antibody linked to the variable region of the selected antibody, the signaling domain of the human CD28 receptor and a transmembrane domain.

9. The recombinant peptide of claim 8, wherein the transmembrane domain to the human CD28 transmembrane domain.

10. The peptide according to claim 9, wherein the selected antibody is an anti-G_{D2} antibody.

11. T cells expressing a recombinant peptide comprising the variable region of the light chain of selected antibody linked to the variable region of the heavy chain of the selected antibody and to the signaling domain of the human CD28 receptor and a transmembrane domain.

12. T cells of claim 1 1, wherein the transmembrane domain to the human CD28 transmembrane domain.

13. T cells according to claim 1 1, wherein the selected antibody is an anti-G_{D2} antibody.

14. T cells according to claim 13, wherein the T cells further express a suicide gene.

15. T cells according to claim 14, wherein the suicide gene encodes thymidine kinase.

16. A method for inducing in a host an immune response to tumor cells expressing a surface antigen comprising the steps of
(a) transducing T cells to introduce an expressible recombinant polynucleotide encoding a fusion protein comprising the variable region of the light chain of an antibody against the surface antigen, linked to variable region of the heavy chain of an antibody against the surface antigen, the signaling domain of human CD28 receptor and a transmembrane domain; and
(b) introducing transduced T cells expressing the recombinant polynucleotide into the host.

17. The method according to claim 16, wherein the transmembrane domain to the human CD28 transmembrane domain.

18. The method of claim 16, wherein the tumor cells express G_{D2} as a surface antigen, and wherein the fusion protein includes the light chain and the heavy chain of an antibody against G_{D2}.

19. The method according to claim 18, wherein the expressible polynucleotide further encodes a suicide gene.

20. The method according to claim 19, wherein the expressible polynucleotide further encodes a suicide gene.
